(19) United States
(12) Patent Application Publication
(10) Pub. No.: US 2002/0111692 A1
(43) Pub. Date: Aug. 15, 2002

(54) Artificial hip having a femoral stem portion which provides for micromovement

(76) Inventors: James D. Ralph, Oakland, NJ (US); Stephen Tatar, Montvale, NJ (US)

Correspondence Address: Joseph P. Errico, 150 Douglas Rd, Far Hills, NJ, 07931, US

(21) Appl. No.: 09/784,273

(22) Filed: Feb. 15, 2001

(51) Int. Cl. A61F 2/36

(52) U.S. Cl. 623/23.17; 623/23.27; 623/23.23

(57) ABSTRACT

A femoral stem for use with an artificial hip joint assembly having helical cuts formed in the shaft thereof. The helical cuts form a restoring force spring-like shock absorbing geometry which permits the rigid metal material to grossly conform to the elastic modulus of the patient's bone. This femoral stem reduces the shock loading and attending pain associated with activities, such as standing, walking, and descending stairs, which often plague hip replacement patients.

### FIELD OF THE INVENTION

This invention relates generally to an artificial hip joint and in particular to the stem portion thereof, wherein the stem portion is designed to minimize the transmission of sharp pressure loadings on the upper portion of the femur by absorbing the energy of the pressure loading, and most specifically to femoral stem portions which comprise a force restoring design, such as a series of helical cuts formed therein.

### BACKGROUND OF THE INVENTION

Artificial devices for replacing degenerated and/or traumatized joints are well known in the art of orthopaedic surgery. Implantable joints comprising ball and socket articulating surfaces, crudely mimicking the natural joint anatomy, are generally utilized in hip and shoulder reconstruction. The ball surface of such an implants is traditionally disposed at the end of an elongate element which is mounted into the end of the long bone associated with the joint such that the ball portion of the structure is directed into the nexus of the joint. The socket portion of the replacement joint is generally formed of a shell and cup combination in which the shell is embedded (by screws, bony ingrowth, and/or bone cement) into the concave recess of the joint. For example, in the case of a hip implant assembly, the ball portion of the joint is mounted to a stem having a shaft and neck, the shaft being inserted axially into the top of the femur. The socket portion of the hip implant assembly is generally inserted into the natural recess of the pelvis (acetabulum) and receives the ball into its concave surface. The socket portion, as introduced above, generally comprises a metallic shell portion and a polymeric (ultra-high molecular weight polyethylene) cup which nests in the shell.

The natural hip joint includes a number of organic elements (cartilage, tendons, ligaments, and musculature) which serve various functions, including cushioning the socket in the event of suden pressure loadings, for example, standing up, jumping, or stepping down a stair. The degeneration of the hip joint may be caused by the breakdown of any of these tissues, and generally results in instability, a restriction in the range of motion, and pain. Unfortunately, in order to replace the degenerated joint, a number of these natural cushioning elements must be removed. Those which are not removed are often traumatized to the point of initiating scar tissue buildup which limits the cushioning of the joint.

The femoral stem of the artificial hip implants presently provided for in the art generally include a metallic shaft which is inserted into the central bore of the femur, after the removal of the diseased femoral head has been achieved. The shaft of the femoral stem is often secured within the bore of the femur by additional artificial means, such as bone cement (hydroxyapatite) or metal pins. Alternatively, the stem may be coated with a porous material which permits the bone to grow into, and fuse with the implant.

The difference in the moduli of elasticity between the metal of the implant and the surounding bone, however, causes a disproportionate, and unnaturally sharp loading of the surrounding bone during actions, such as stepping down a stair or standing up quickly. These sharp loadings shear the microinfusions of bone into the femoral stem, or jar the socket so sharply as to cause an excrutiating pain to rifle through the patient's leg and hip. This failure of implants of the present state of the art has been recognized by many as being the result of the disjunction in the modulous of elasticity of the implant as compared with the bone. Modest attempts at alloying different metals to bring the implant's elasticity closer to that of bone have been made. The vast difference between metal and bone, however, have made this a fruitless endeavor. Further, such alloying attempts, were they to be even moderately successful, would fail in the majority of patients, as the variation of bone strength within the classes of patients would lead only some to be relieved.

It is, therefore, a principal object of the present invention to provide an implantable femoral stem which provides widespread alleviation of the severe pain associated with hip implant devices when sharply loaded by normal activity.

In addition, it is a further object of the present invention to provide an implantable hip assembly in which the conformation of the hip stem provides a cushioning for the sharp loadings associated with normal activity.

### SUMMARY OF THE INVENTION

The preceding objects are provided in the present invention which is a new and novel artificial hip implant having a femoral stem member in which the bone shaft portion of the stem comprises at least one section which is helically cut. More specifically, the at least one helically cut portion of the shaft shall be cut such that the stem has a spring-like force restoring capacity and such that the loading of the surrounding bone is maintained at a more typical level during normal activity. In certain preferred embodiments, the helically cut shaft shall further be coated and/or sheathed in a plastic covering to which the adjacent bone may adhere and/or fuse with, but which ingrowth will not interfere with the micromovement of the stem itself.

In alternative embodiments which may be contemplated, a seating member may be incorporated with the stem member, through which seating member the stem may be inserted into the top of the femur, and the femur may grow into the seating member and not into the stem directly. In such an embodiment, the stem member is securely coupled to the seating member once the assembly is fully implanted. A helically cut portion of the stem would therefore be provided between the locking interface of the seating member and the ball head of the stem.

More particularly, with respect to the general concept of the present invention, the generic geometry of a spring is particularly suited for the present application in that it permits the designer to change the elastic modulous of the device without having to modify the material characteristics of the metal being used. Helical cuts provided along the shaft of the stem geometrically effectively extends the axial dimension through which elastic compression may occur is enhanced while not altering the overall length. In addition, the effective angle of loading permits a lateral deflection of the metal, as opposed to an axial one. These two geometric factors allow the same material to compress more, under the same stress, than a solid member would.

Referring again to the potential embodiments of the present invention, two classes of potential device are identifiable; the classes being distinguishable relative to the axial positioning of the helical cuts. The first class of micromovement femoral stem members includes the cuts at the upper portion of the stem, between the portion which is inserted into the top of the femur and the ball head of the stem. The second class incorporates the cuts at the distal tip of the shaft, deep within the femoral bone. As introduced above, the first class of embodiments may include a seat member which is mounted in the top of the femur; or it may be directly secured to the femur. The second class of embodiments is more suited for direct implantation into the femur, i.e. without an additional seating member. Both classes, however, may include a polymer shealth disposed about the helically cut section to permit free mobility, unencumbered by bone ingrowth.

In combination with a generic artificial hip implant design, the various embodiments of the present invention shall be set forth in the descriptions to followin conjunction with an acetabular cup member, including a socket portion and a shell (the socket of the ball and socket joint), as well as the aforementioned stem member which includes a helically cut shaft and an upper rotating surface (the ball of the ball and socket joint). The associated methods of implantation are also more fully set forth hereinbelow in conjunction with the Figures provided herewith.

### BRIEF DESCRIPTION OF THE DRAWINGS

FIGS. 1a and 1b are side views of a femoral stem seating member and a helically cut femoral stem member of the present invention, respectively;

FIG. 2 is a side view of another femoral stem having a helically cut distal end of the shaft portion, which is an aspect of the present invention;

FIGS. 3a and 3b are perspective views of an acetabular shell and cup assembly, respectively, which may used in conjunction with either femoral stems of the present invention;

FIG. 4 is a side cross section view of a fully assembled hip implant embodiment which is an aspect of the present invention including the stem and seating member as set forth in FIGS. 1a and 1b; and

FIG. 5 is a side cross section view of a fully assembled hip implant embodiment which is an aspect of the present invention including the stem as set forth in FIG. 2.

### DETAILED DESCRIPTION OF THE PREFERRED EMBODIMENT

While the present invention will be described more fully hereinafter with reference to the accompanying drawings, in which particular embodiments and methods of implantation are shown, it is to be understood at the outset that persons skilled in the art may modify the invention herein described while achieving the functions and results of this invention. Accordingly, the descriptions which follow are to be understood as illustrative and exemplary of specific structures, aspects and features within the broad scope of the present invention and not as limiting of such broad scope. Like numbers refer to similar features of like elements throughout.

Referring now to FIGS. 1a-b and 2, the present invention comprises a femoral stem which includes a shaft portion having a proximal end and a distal end, said distal end being shaped for insertion into the axial bore of a patient's femor. This stem further includes, at its proximal end, a ball-shaped surface for insertion into a receiving cup disposed in an acetabular recess in said patient's pelvis. The novel feature of the present invention is the inclusion of at least one helical cut formed in the shaft portion of the stem. The helically cut segment of the shaft portion is thereby endowed with an elastic modulous which is substantially less than that of the remaining portions of the stem, and which elastic modulous approximates that of the patient's bone. The geometry of the helical cuts and the specific positioning of the cuts are maybe provided in various manners which are solely engineering expediencies.

In particular, in a first embodiment illustrated in FIG. 1a, the stem 100 comprises a shaft portion 102 and a ball-shaped head 104 disposed at the proximal end 106 of the shaft. The helically cut segment 108 of this first embodiment is disposed between the proximal and the distal ends of the shaft. More specifically, the position of the helical cut corresponds to the seating element shown in FIG. 1a, which may be used in conjunction with this first embodiment. The seating element 110 comprises a frusto-conical tubular member having a proximal 112 and a distal 114 end. The interior tubular surface 116 has a distal portion 118 which is tapered such that it narrows in the direction of the distal end. The distal end 114 of the seating member 110 is insertable into an axial bore of a patient's femor.

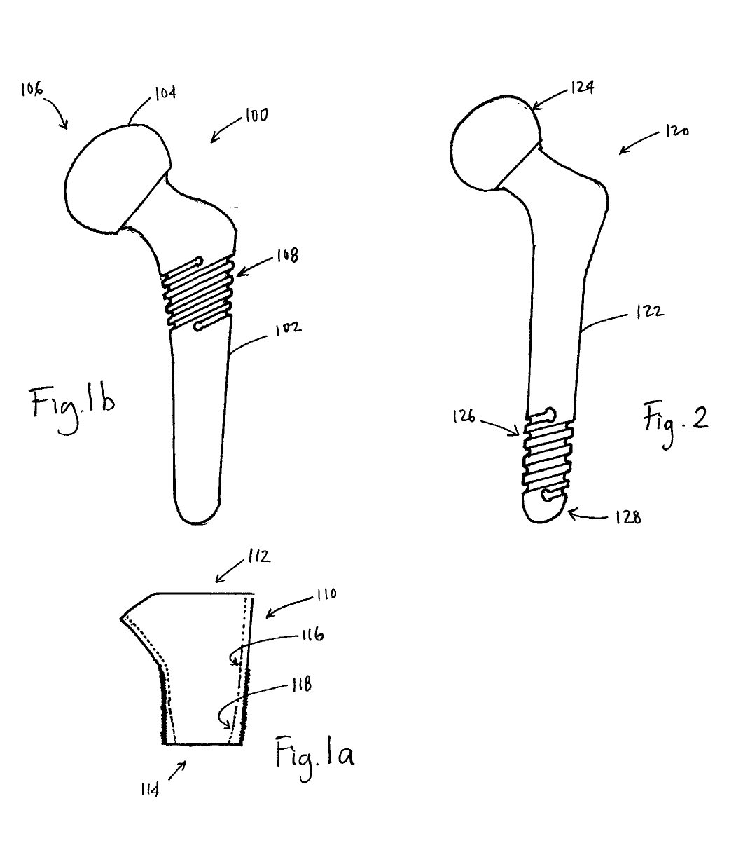

An alternative embodiment of the present invention is shown in FIG. 2. As with the stem shown in FIG. 1a, this stem member 120 comprises a shaft portion 122 having a proximal end and a distal end, said distal end being shaped for insertion into the axial bore of a patient's femor. This stem 120 further includes, at its proximal end, a ball-shaped surface 124 for insertion into a receiving cup disposed in an acetabular recess in said patient's pelvis. Again, the novel feature of the present invention is the inclusion of at least one helical cut 126 formed in the shaft portion of the stem. In the second embodiment, the helically cut segment of the shaft portion is provided at the distal end 128 of the shaft.

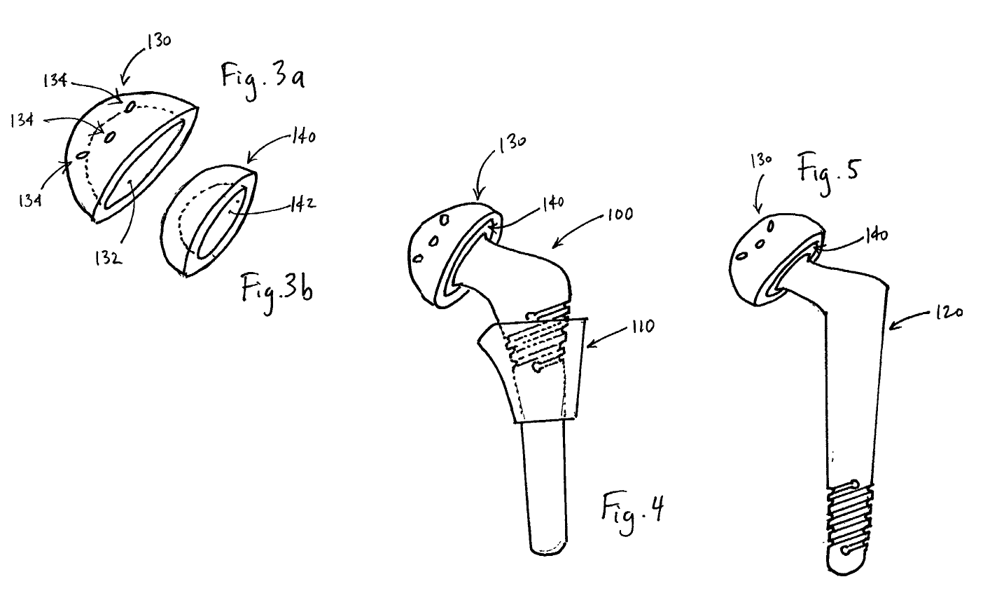

The stem members described above include a ball-shaped head which serves as the ball of the ball and socket joint replacement. The acetabular shell and cup portions shown in FIGS. 3a and 3b, comprise the elements of the socket portion of the artificial joint. More particularly, the shell 130 is a hemispherical element having a concave recess 132 formed therein. Acetabular shells of the type shown here are generally constructed of a medical grade metal, such as stainless steel, cobalt chrome, or titanium. The metals are selected because of their superior fatigue and strength characteristics, as well as their tisue compatibility. A series of screw holes 134 may be provided for securing the shell within the prepared site of the acetabulum of the patient. Alternatively, bone growth coatings and bone cement may be utilized to secure the shell 130 in place.

FIG. 3b shows a cup element 140, generally made of a polymer, such as polyethylene, or of a polished ceramic. The cup 140 is a hemispherical element, the exterior surface of which seats into the recess 132 of the shell 130. The cup 140 also includes a recess 142, into which the ball-shaped head of the stem portion seats and rotates.

With reference now to FIG. 4, in which a fully assembled first embodiment of the present invention is provided, methods of implantation are herein described in such a manner as will be illustrative of the advantageous features of the helically cut stem. More particularly, the orthopedic surgeon begins the joint replacement by preparing the joint site for receiving the femoral and acetabular components. Specifically, the head of the femur is removed and the seating member 110 is is mounted in the femur. The stem portion 100 of the femoral implant is then inserted into the axial bore of the seating member until the intermediate portion of the shaft comes to nest against the tapered interior surface of the seating member. The helically cut portion of the shaft is disposed between the seating member and the ball-shaped head, such that shock loading of the shaft will be absorbed by the intermediate portion of the stem with the same, or similar, elasticity as the femoral bone.

The shell 130 is then inserted into the acetabulum (which is prepared by clearing the cartilage and other tissues disposed above the bone). Once the shell 130 is secured in the acetabulum, via screws, cement, or other equally effective fixation means, the cup 140 is inserted into the recess in the shell such that the cup the head of the femoral component are aligned properly.

Referring now to FIG. 5, a fully assembled view of the second, and alternative embodiment of the present invention is provided. In this second embodiment the seating member is not necessary, and the force absorbing helical cut of the stem is disposed at the distal tip of the shaft.

While there has been described and illustrated specific embodiments of new and novel ball and socket implant devices, it will be apparent to those skilled in the art that variations and modifications are possible without deviating from the broad spirit and principle of the present invention which shall be limited solely by the scope of the claims appended hereto.

We claim:

1. A femoral stem member for use in an artifical hip implant assembly, comprising:
a shaft portion having a proximal end and a distal end, said distal end being shaped for insertion into the axial bore of a patient's femor, and said proximal end including a ball-shaped surface for insertion into a receiving cup disposed in an acetabular recess in said patient's pelvis;
at least one helical cut formed in said shaft portion such that said helically cut segment of the shaft portion has an elastic modulous which is substantially less than that of the remaining portions of the stem, and which elastic modulous approximates that of the patient's bone.

2. The femoral stem member as set forth in claim 1, wherein said at least one helical cut includes a helical cut formed in a segment of the distal end of the shaft portion.

3. The femoral stem member as set forth in claim 1, wherein said at least one helical cut includes a helical cut formed in a segment of the proximal end of the shaft portion adjacent to the ball-shaped surface.

4. The femoral stem member as set forth in claim 1, wherein at least one of said helically cut segments further includes a polymer sheath surrounding said cut, which polymer sheath prevents the patient's bone from ingrowth into the helical cuts, thus preventing interference with the geometric modification of the overall modulous of elasticity of the helically cut segment.

5. A femoral stem and seating member assembly, for use in an artificial hip implant, comprising:
a frusto-conical tubular member having a proximal and a distal end, said member having an interior tubular surface, at least a portion of which is tapered such that it narrows in the direction of the distal end, said distal end of said frusto-conical member being insertable into an axial bore of a patient's femor;
a shaft member having a proximal portion, an intermediate portion, and a distal portion,
said distal portion being shaped for insertion through the frusto-conical tubular member and into the axial bore of the patient's femor,
said intermediate portion being tapered to seat against the narrowed interior tubular surface of said frusto-conical tubular member,
said proximal end including a ball-shaped surface for insertion into a receiving cup disposed in an acetabular recess in said patient's pelvis;
at least one helical cut formed in said shaft portion such that said helically cut segment of the shaft portion has an elastic modulous which is substantially less than that of the remaining portions of the stem, and which elastic modulous approximates that of the patient's bone.

6. The femoral stem and seating member assembly as set forth in claim 5, wherein at least one of said at least one helical cut is formed at the interface between the proximal and the intermediate sections of said shaft member.

7. The femoral stem and seating member assembly as set forth in claim 5, wherein said frusto-conical tubular member includes a surface coating which is osteoinductive.

8. The femoral stem member as set forth in claim 5, wherein said at least one helical cut further includes a helical cut formed in a segment of the distal end of the shaft portion.

9. The femoral stem member as set forth in claim 5, wherein at least one of said helically cut segments further includes a polymer sheath surrounding said cut, which polymer sheath prevents the patient's bone from ingrowth into the helical cuts, thus preventing interference with the geometric modification of the overall modulous of elasticity of the helically cut segment.

10. A hip implant assembly comprising:
a socket portion positionable within an acetabular socket of a patient's hip;
a frusto-conical tubular member having a proximal and a distal end, said member having an interior tubular surface, at least a portion of which is tapered such that it narrows in the direction of the distal end, said distal end of said frusto-conical member being insertable into an axial bore of a patient's femor;
a shaft member having a proximal portion, an intermediate portion, and a distal portion,
said distal portion being shaped for insertion through the frusto-conical tubular member and into the axial bore of the patient's femor,
said intermediate portion being tapered to seat against the narrowed interior tubular surface of said frusto-conical tubular member,
said proximal end including a ball-shaped surface for insertion into a receiving cup disposed in an acetabular recess in said patient's pelvis;
at least one helical cut formed in said shaft portion such that said helically cut segment of the shaft portion has an elastic modulous which is substantially less than that of the remaining portions of the stem, and which elastic modulous approximates that of the patient's bone.

11. The femoral stem and seating member assembly as set forth in claim 10, wherein at least one of said at least one helical cut is formed at the interface between the proximal and the intermediate sections of said shaft member.

12. The femoral stem and seating member assembly as set forth in claim 10, wherein said frusto-conical tubular member includes a surface coating which is osteoinductive.

13. The femoral stem member as set forth in claim 10, wherein said at least one helical cut further includes a helical cut formed in a segment of the distal end of the shaft portion.

14. The femoral stem member as set forth in claim 10, wherein at least one of said helically cut segments further includes a polymer sheath surrounding said cut, which polymer sheath prevents the patient's bone from ingrowth into the helical cuts, thus preventing interference with the geometric modification of the overall modulous of elasticity of the helically cut segment.
